(19) United States
(12) Patent Application Publication
(10) Pub. No.: US 2005/0042741 A1
(43) Pub. Date: Feb. 24, 2005

(54) Stenoprophiluric generation and isolation of chemical products

(76) Inventor: Dennis A. Guritza, Chagrin, OH (US)

Correspondence Address: HAHN LOESER & PARKS, LLP, One GOJO Plaza, Suite 300, AKRON, OH, 44311-1076, US

(21) Appl. No.: 10/503,524

(22) PCT Filed: Feb. 5, 2003
(86) PCT No.: PCT/US03/03311

Related U.S. Application Data
(60) Provisional application No. 60/354,904, filed on Feb. 5, 2002.

(51) Int. Cl. C12M 1/34

(52) U.S. Cl. 435/287.1

(57) ABSTRACT

Stenoprophiluric media provide for the creation and sustained well being of micro-habitats in uncontrolled host habitats in order to study the biological, biochemical and physical (morphological) properties of the organisms and their consortiums inherently and/or in relationship to competing or proximal micro-habitats as well. Given this in-situ method of study of these consortiums; sampling of metabolic bio-chemicals both primary and secondary is readily achieved. By providing this medium or platform for natural in-situ evaluation of varying host habitats and/or multiple sub-habitats (micro-habitats) in the same host habitat, conditions and changing relationships can be evaluated; physically, biologically, and chemically. Evaluation enhancements by this method provides for the ability to sample discrete chemicals or groups of chemicals and/or compounds for easy bio-activity evaluation. Further, morphological and taxonomic evaluation of micro-habitats is permitted whereby, relationships and evaluation of changes in settling, growth, or mature bio-film formations can be readily accomplished.

This application claims priority from U.S. provisional patent application Ser. No. 60/354,904, which is incorporated by reference as if fully recited herein.

The present invention relates to a device and a process for eliciting the production of chemical products by a biologically active agent due to the proximity of the biologically active agent to a stimulative agent in a host habitat. In the present invention, the biologically active agent is maintained in a stenoprophiluric medium in the host habitat.

### BACKGROUND OF THE ART

Naturally derived chemical compounds as drugs and medicinal treatments have been used by shamans and medicine men for centuries. Modem society, seeking wider distribution and safe use of these bio-active materials, requires isolation, preparation, and the appropriate development of use criteria. While specific organisms both plant and animal have long been associated with the production and effects of theses materials, the methods used to collect and bio-reactivity screen test have been painstakingly slow and costly to develop. In the late 1950's, the National Cancer Institute sponsored a mass screening of plant materials which became the first large scale search method to be used. Most of the early efforts were initially aimed at temperate forests or other land based ecosystems. One of the first discoveries from a temperate forest was Taxol, a drug effective against several forms of cancer. The chemical compound from which Taxol was identified is an extract from Pacific Yew tree. This discovery occurred in 1963. However, it was not until 1993, following an arduous path of trials and synthesis evaluations, that the drug was finally approved for pharmaceutical production. Typically, one major requirement for success to be a candidate commercial compound is the need to be synthesizable. Otherwise, farming or culturing would be the only source of the needed quantities of the chemical. Since the amount of active material Taxol (derivative compound) in the actual Yew tree is very small relative to the size or mass of the tree, farming is not practical. So, proof of a viable synthetic route was required to sustain continued investment in the pending product. Consequently, alternative production methods (farming, reactor generation or chemical synthesis) of bio-active chemical compound candidates are always sought from natural compound sources to insure a supply of active compounds that meet all the required criteria for commercial success.

Many years after Taxol, Bryostatin-1 was discovered. Bryostatin-1 is an allelopathic chemical associated with a very specific marine organism. The organism in this case is a bryozoan Bugula neritina, resembling a spiny or hard sponge. When this bryozoan is in the presence of a bacteria (Candidatus Endobugula sertula), the bio-active allelochemical Bryostatin-1 is produced. This discovery renewed interest in allelochemicals from the sea. Once again, however, finding the compound while very positive in itself, found new hurdles to production and commercialization. While farming was believed to be the probable source of material for commercial consumption, the only known place where this chemical is naturally produced is at a specific location of temperate waters off of southern California. This made farming a very remote possibility for creating a sufficient commercial supply. Fortunately, a synthetic form, Bryostatin-2 has been formulated. Two issues are thus illustrated. First, the illusiveness of random identification of viable compounds in the wild. Second, if the compound can not be synthesized or economically farmed it may have very limited commercial value. Particularly if natural habitats can not produce sufficient supply. This discovery while helping to renew the enthusiasm for aquatic, particularly marine, candidates serves as an impetus to widen the search for new natural chemical compounds. However final production sourcing and the cost of random searches for natural compounds still posses great cost and technical barriers in front of the researchers. Increased demand for new compounds in light of current crises in lack of effectiveness of “old stand-by anti-biotics” establishes the need to review many more viable candidates at a much lower cost.

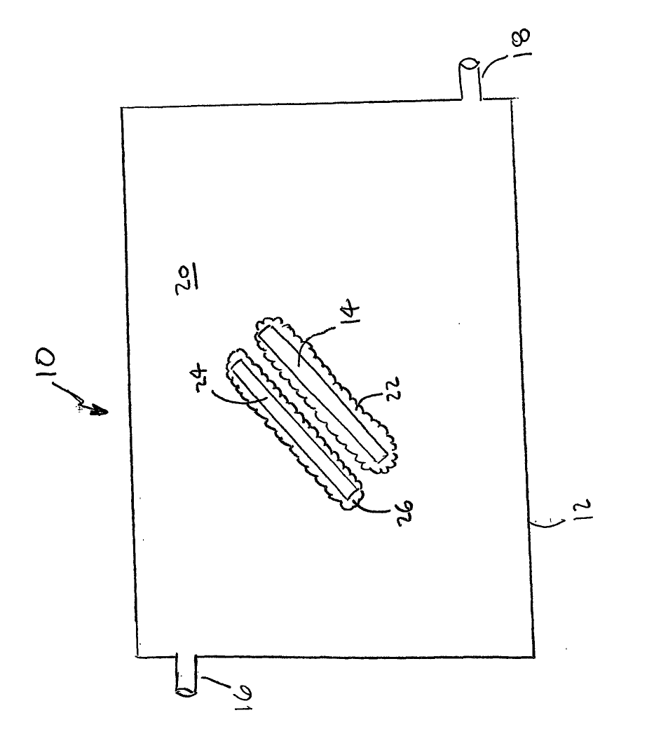

The marine environment has held the promise for new bio-reactive chemicals and pharmaceuticals since 1896 when Apstein linked allelochemicals (chemicals produced by organisms for use outside the cell) to the specific definition of community structure. Dr. Luigi Provasoli dedicated a lifetime of work attempting to define the parameters necessary for laboratory (in vitro) media which could permit the isolation and potential production of allelochemicals. His dedicated efforts were aimed at unlocking pharmacological uses of allelochemicals as anti-biotics. Dr. J. William Costerton, University of Montana, Bozeman, has recently illustrated and photographed the relationships of multiple organisms in heretofore unrecognized micro-communities with new methods in confocal scanning laser microscopy. (http://www.asmusa.org/edusrc/biofilms/index.html) This new technique illustrates the intricacies of micro-habitats where bacterial symbionts form slime communities for the well being of themselves and their host. (See FIG. 1) Dr. George Petit, Wayne State University, directed a team to the discovery of Bryostatin, a synthesizable new drug, from a marine invertebrate and its bacterial symbiont, with potent anti-tumor, leukemia, and extensive anti-cancer applications. (http://www.acdlabs.com/webzine/1/1_{—}1f.html) Dr. K. Irwin Keating in her extensive writings has provided a focus on the potential for deriving many new anti-biotics from allelochemical processes which occur in nature on a regular basis. Dr. Keating also has related the enormous implications from a comprehensive understanding of intricate allelochemical relationships and the benefits of understanding other inter-cellular bio-chemical processes as well. (http://snowfall.envsci.rutgers.edu/˜kkeating/cv_html) Dr. Alicja Zobel, Trent University, Peterborough, Ontario, challenges colleagues to seek an understanding of routes, relationships, pre-cursors and the identification of naturally occurring bio-chemical agents. The intent being the prevention of disease through the positive stimulation of natural immune systems via nutritional adjustments and supplemental additives to diet, which could be found in review of natural compounds. Dr. Zobel is concurrently seeking allelochemicals and other natural metabolites as new candidates in the treatment regime for fighting diseases such as cancer, aids, and melanomas, in an effort to add to the current arsenal of treatments. (http://www-ias.uca.es/zobel.html) The incentives for understanding the bio-chemistry of natural systems as source compounds are tremendous. The natural interactive abilities of organisms are just now being brought into focus by international scientists with the belief that allelopathy holds great promise in pharmacology, and replacement of man-made herbicides and pesticides. While much of the early focus was on forests, marine habitats, with an order of magnitude greater likelihood of success than land based (terrestrial) habitats has always attracted the scientific community with a much higher degree of interest. Other, equally exciting opportunities for research in aquatic allelochemicals include new chemical compound solutions for fundamental industrial and agricultural problems as well. Oil and fuel biocides, non-toxic closed loop water systems, and even the field control of the parasite responsible for schistosomiasis are to name but a few.

In other areas of science in recent years, work in settling characteristics of micro-organisms on substrates, the role of nutritional components by species and availability on sustained biological communities, and inter-element/compound effects as metabolic regulators as well as becoming building blocks for primary and secondary compound synthesization have been defined. See, for example, Bacterial Adhesion. Mechanics and Physiological Significance. Savage, D. C. and Fletcher, M., Eds., Plenum Press, New York. 1985, and Bacterial Adhesion. Molecular and Ecological Diversity. Fletcher, M., Ed., Wiley-Liss, New York, 1996. Community maintenance and availability of nutrients and agents for controlling a micro-habitat can be sustained through assuring a regular ability of interaction between the substrate (Stenoprophiluric media) and the adhered bio-mass/bio-film consortium.

Bacterial adhesion and molecular and ecological diversity has been described in the literature primarily as it relates to inert or nearly inert substrates. This would be where the substrate is primarily incidental or only initially interactive with a resulting biomass or the substrate is a material which is introduced into a host habitat where the natural environment opportunistically attacks, consumes, or degrades the substrate for food or simply corrodes the substrate material due to chemical incompatibilities. From these studies it is understood that composition does in fact influence settling in several ways; chemically, electrically, physically, and in terms of nutritional sources. “Settling” is simply defined as the first of a three stage evolution of organisms collecting on a surface. Stage one (settling) which is reversible is where organisms from the host habitat, typically in a floating or planktonic state find favor in landing on a surface for adhesion either due to electrical force interaction, nutritional source attraction, or source ques for essential nutrients or compounds which are integral to any of the metabolic functions of the organism. From the electrical perspective in early settling a substrate may contain a “grid-like” pattern based on the substrate composition and configuration which facilitates orientation of an organism and results in a “settling” pattern derived from the substrate properties. Preferential selection of one organism over another or providing for the attachment opportunity which does not exist in the host habitat in order for the organism to affect metabolic changes often associated with growth and reproduction occurs and is influenced by these surface effects. Stage two is when an organism species or consortium of species determines that favorable conditions exist for changing from a free floating or planktonic state to a more sessile or attached or adhered state, where explosive or trigonometric growth in the numbers of organisms on the surface or substrate occurs. During this stage, the number of organisms dramatically increase, typically associated with production of exo-polymer to support adherence and protection of the new consortium. The third stage is the mature stage where in an overly simplified sense, the consortium goes into a space management capability control phase or develops a secondary or gradient ecology where development of highly specialized morphological relationships arise with a consortium providing very complex relationships of nutrition, waste management and spatial protection as just a few examples of symbiotic relationships that are developed and given sufficiently sustained nutritional support by regulatory elements and compounds can sustain the community for extended periods of time or at least as long as the supply of the essential components is available from the substrate or the host habitat. Interestingly in other areas of science in dealing with potential toxic effects of excess amounts of effluent containing such thongs as heavy metals or other organic “pollutants” the general consensus of many of the authors is that bioavailability and toxicity both are regulated by the species and concentration of any subject compound in question. Consequently, compounds and elements available in consortiums or gross biomass communities find that the concentration and species of such things as nutrients, toxics, and bio-active agents are critical to there success as a consortium and even as to their actual survivability. The robustness to establish a community and then to maintain it is based on spatial favor-ability of settling and maturing into a specific biomass and the dependency on nutritional and chemical supplies to provide for primary metabolic or secondary metabolic functions is directly related to supply of those materials in the correct time sequence, species of element or compound at a desired concentration, and its sustained availability. That these materials contribute to the capacity of the organism(s) to metabolically produce needed compounds for growth and reproduction and the ability to formulate secondary metabolites used for communication, allelopathy, or other means of protecting themselves or their space of attachment has been made clear. At the same time, as has been witnessed by Provasoli and many others, “in-vitro” conditions can rarely match “in-situ” conditions such that the result metabolism, biochemistry, ro growth patterns experienced in natural host habitats. The first question then becomes can we understand these relationships in a consortium community and how it relates to managing its relationship to other communities, then the second question becomes can these chemicals and compounds be used in other situations beneficially. Historically the focus on reproducing natural events in the lab or “in-vitro” has led to some gereat discoveries. However, truly understanding the relationships of the organisms to themselves, their micro-habitat consortium, and their capacity to provide for a complete set of communication and functional chemical events is dependent on sufficient quantity of the biomass resident in its host habitat to permit the observations, sampling and causal effects that occur in these distinct ecological niches.

### Barriers to Natural Compound Discovery

One of the largest barriers to successful discovery of the function and potential alternate use of these natural chemicals has been in defining a methodology with the ability to relate the function of the allelochemicals or other metabolites to the organisms natural systems as found in micro-habitats(in situ), in the wild. Attempting to observe a chemical interaction in isolation on a reef with thousands of other events occurring simultaneously is an extreme, often impossible challenge. Alternatively, bringing selected organisms to the laboratory and encouraging them to “act naturally” is even more difficult. Duplication of natural interactions in the lab would be certainly possible if all of the important natural life support parameters were known. Unfortunately, we do not know all of these parameters. Consequently, the discovery of a new method which utilizes controlled micro-habitats dramatically changes and greatly enhances the opportunity for identifying and verifying new uses of allelochemicals. By understanding natural mechanisms, we can look to “bio-mimicry” as a viable solutions to many problems. With the ability to facilitate discreet sampling and identification of chemical compounds in direct correlation with the organisms based on known activity “trigger responses” which can be produced in a natural setting would make for a new dimension of facilitated study. This radically new approach will permit scientists to understand the purpose for which the allelochemicals are intended and focus on the relationships as well as the chemistry. Then isolation of those compounds for review can be achieved, including the recognition of any synergistic effects with combinations of chemicals in the specialized micro-habitat.

### History of Methodologies

Taxol is an example of the “mass screening” procurement method of plant material initialized by the National Cancer Institute in 1963. (www.taxol.com/txstory.html) The Taxol story is a result of proverbially taking the “hay stack” grinding it up, separating it into its surviving component parts and looking at each “needle” in isolation from its history or suspected allelochemical purpose. Using this random search method, with each of the 100,000 needles you distinguish, you may get statistically one candidate for serious clinical trials. While this method has been a primary method of collection for many years, it has obvious inherent severe limitations. The most obvious being cost and screening capability. To stay with the analogy, some needles are destroyed in the process, and some simply get lost or are not properly recognized in the screening process. Following its isolation as a distinct compound, a successful candidate must then survive synthesization, application criteria development, and the myriad of production and approval parameters. Each of these steps having their own varying degrees of uncertainty and substantial investment risk from the business perspective. While intriguing to biologists and chemists, investors and corporations view the return on massive investment with great reluctance. Additionally, it has only been in the last several years that advances in analytical equipment and rapid screening of multiple compounds for analysis has developed sufficiently to handle many of the fragile chemicals being collected.

The second and distinctly different approach taken by biologists has been in attempting to develop cultures of “critters” which are hopefully isolated down to an empirical level in order to facilitate the reproduction of discreet chemical relationships in the laboratory (in vitro). Re-creation of these natural events in the lab also has found very limited success. But as a method, attempts continue on a regular basis. As early as 1940 with Pratt's isolation of an anti-biotic called Chlorellin, which is produced by a form of algae, the development of cultures which could produce these potential drugs has been sought. Systematically, the discovery of the complexity and lack of a complete understanding of the pathways of natural synthesis of these chemical components along with the probability of both sequential and hierarchical reactions has stymied success. Duplicating natural conditions in the lab while theoretically possible is seldom achievable. Lacking a single micro-nutrient may render a complete system totally in-operable.

The third methodology which has emerged and has been driven by the success of Taxol and Bryostatin, in light of the time and cost shortfalls as well, has been “selective sampling.” By knowing or suspecting a source of new compounds, collection and selective screening occurs. However, with the complicating factors of complex ecosystems in the wild, the time line can be as long as taking the mass screening approach, with the odds being a lot riskier. Having fewer potential “candidates” in the pipeline makes success far less probable than in the mass screening approach. Logistic and geographical issues also intervene. As in the case of Bryostatin, fourteen other global locations were chosen for sampling, in the event that farming for chemical production would be required. Each location was known to have the selected bryozoan, however, no Bryostatin was found. The complexity of the habitats and/or the inherent nature of “event” sampling intervened. When bio-reactive candidates are found they need to be either farmed or synthetically produced in sufficient affordable amounts to justify commercialization.

Yet the need for new sources of drugs reaches higher levels of urgency every day. In 1994, Begley indicated that hospital infections caused by Staphylococcus are becoming increasingly more resistant to traditional antibiotics. At the same time Begley indicates that antibiotics from marine organisms hold perhaps the best promise. Unfortunately, if we continue with old methodologies like mass screening, the hay stack is too big. Lacking a more educated search on focused targets and a viable affordable methodology we do not have the time or dollars for a comprehensive search. We then fail to meet the huge demand. From the perspective of the methodology which attempts to isolate cultures in the lab (in vitro), similar lack of success has been found with little hope for breakthroughs. Entire production facilities devoted to culture production have been constructed, in recent years, only to find that the process doesn't work. Some of the related problems with this culture approach include; interference by other organisms (contamination), a lack of transfer of any symbiont relationships in the culture, the absence of bio-chemical triggers, inappropriate environmental conditions, and the lack of correlation of probable synergies involving more than one organism. For example, Costerton and his associates have pointed out in their work that it is five specific bacteria in concert with each other are responsible for making cellulose digestion in cows viable. The symbionts (bacteria) in the slime layer provide the physical and chemical micro-habitat to facilitate the digestion of cellulose, one of the most difficult digestion processes known. Facilitating the creation and understanding of these specialized micro-habitats does not occur when viewed by a method using isolated cultures.

From the foregoing, it is clear that there is a long-felt need to provide a device and a process to generate and isolate particular desirable chemical products from a biologically active agent.

### SUMMARY OF THE PRESENT INVENTION

This object and other objects are achieved by a device for isolating a chemical product generated by proximity of a reactive biological agent to a stimulative agent. Such as device has a container with an inlet and an outlet. A host habitat fluid flows through the container from the inlet to the outlet. In the container, at least one first substrate is supportably positioned between the inlet and the outlet, preferably directly in the flow of the host habitat fluid. A stenoprophiluric medium is disposed on the first substrate. Such a stenoprophiluric medium comprises a bio-supportive medium, with at least one layer there of the medium deposited on of each of the first substrates. The bio-supportive medium itself comprises a degradable material, at least one nutritional source and at least one bio-limiting agent dispersed in the degradable material. The degradable material, the at least one nutritional source, and the at least one bio-limiting agent are provided in quantities, such that the bio-supportive medium can support formation and development of a biomass of the reactive biological agent in the bio-supportive medium. The biomass has at least a specific consortium of organisms of the same or different species substantially at equilibrium within its environment or host habitat fluid, and the at least one bio-limiting agent is selected to control the amount and type of species present in the biomass. At least one stimulative agent is present in the container, the stimulative agent selected such that proximity of the stimulative agent to the biomass elicits production of the chemical product by the reactive biological agent.

In one embodiment of the invention, the chemical product is dispersed by the reactive biological agent into the host habitat fluid. In such a case, a means for collecting and isolating the chemical product from the host habitat is provided at the outlet of the container.

In another embodiment of the invention, the chemical product is concentrated in organelles of the biomass as it is generated, instead of being dispersed into the bulk host habitat fluid. Inevitably, this portion of the biomass sloughs off from the bio-supportive medium. In such a case, a means is provided at the outlet of the container for collecting and isolating this portion, which may be subsequently processed to recover the chemical product contained therein.

In some of either of these embodiments, the stimulative agent is present in the host habitat fluid, and may be replenished as necessary.

In other embodiments, the stimulative agent is not introduced into the container through the host habitat fluid, but is instead introduced through a second substrate, which is also supportably positioned in the container between the inlet and the outlet. Preferably, the second substrate is movable in the container relative to the first substrate. In one of these embodiments, the stimulative agent is affixed to the second substrate and is released therefrom in a time-controlled manner. In another of these embodiments, the stimulative agent is dispersed from a second bio-supportive medium, which has at least one layer thereof deposited on the second substrate. The second bio-supportive medium is also preferably a stenoprophiluric medium.

In some of the embodiments, the first substrate is at least one plate.

In other embodiments, the first substrate is a cylinder.

In yet other embodiments, the first substrate is a wall of the container.

Other objects of the present invention are achieved by a process for isolating a chemical product generated by proximity of a reactive biological agent to a stimulative agent. The first step of such a process is providing a host habitat fluid in a container having an inlet and outlet, such that the host habitat flows from the inlet to the outlet. The next step is positioning at least one first substrate in the container between the inlet and the outlet, the at least one first substrate having a bio-supportive medium. At least one layer of the bio-supportive medium is deposited on the first substrate. The bio-supportive medium comprises a degradable material, at least one nutritional source and at least one bio-limiting agent dispersed in the degradable material. The degradable material, the at least one nutritional source, and the at least one bio-limiting agent are provided in quantities, so the bio-supportive medium can support formation and development of a biomass of the reactive biological agent therein. The biomass has at least a specific consortium of organisms of the same or different species substantially at equilibrium within its environment or host habitat fluid, wherein the at least one bio-limiting agent is selected to control the amount and type of species present in the biomass. An additional step is to provide further amounts of the at least one nutritional source to the biomass through the host habitat fluid, in order to sustain the biomass. The next step is to introduce at least one stimulative agent in the container. The stimulative agent is selected so proximity of the stimulative agent to the biomass elicits production of the chemical product by the reactive biological agent. The last step is to collect and isolate the chemical product at the outlet of the container.

In some embodiments of the process, the chemical product is dispersed by the reactive biological agent into the host habitat fluid. In such an embodiment, the collecting and isolating step removes the chemical product directly from the host habitat fluid.

In other embodiments, the chemical product is concentrated in the biomass as it is generated, instead of being dispersed into the bulk host habitat fluid. As portions of the biomass inevitably slough off of the bio-supportive medium, they are removed from the container through the outlet in the host habitat fluid. In such a case, the collecting and isolating step involves collecting and isolating the portion of the biomass, which may be subsequently processed to recover the chemical product contained.

### BRIEF DESCRIPTION OF THE DRAWINGS

Better understanding of the present invention will be had when reference is made to the accompanying drawings, in which identical parts are identified by identical part numbers and wherein the single figure shows a schematic view of a reaction system comprising the present invention.

### DETAILED DESCRIPTION OF A PREFERRED EMBODIMENT

Stenoprophiluric media, by being able to select or determine the participation of consortium members in a resulting biomass or bio film, have the potential of providing a sustained specific micro-habitat which may be selected for properties which facilitate the study and collection of compounds, extra-cellular metabolites, and intercellular metabolites which may have beneficial biologically active properties. These micro-habitats and their resulting organism consortiums may be used for, among other applications searching for new and novel compounds for use as drugs and pharmacological agents. By producing micro-habitat consortiums on substrates which are different and perhaps competing in the host habitat, bringing them into contact can trigger allelochemical and other biological responses which produce compounds in single or in combination to provide the capability to evaluate bioactivity in many distinct applications which may be then identified and assessed for multiple reasons. Stenoprophiluric media can be used to determine settling patterns, control second stage growth rates based on bio-limiting Agents or nutrients, and finally, may determine mature bio-film or biomass consortium participants be providing or eliminating specific nutrients in the media. By pre-selection of the nutrient/agent package all stages of a bio-film/biomass can be influenced. Spatial size communities can be created for “farming”, long term growth and metabolic functions can be monitored, and long term conditions supporting such communities can be maintained.

Secondary agents (in stenoprophiluric media) may also be introduced to provide for the ability of the media to enhance a second or subsequent tier(s) of additional organisms in a resultant bio-film or biomass community by providing essential or trace nutrients required for the second tier to prosper in the consortium bio-film either by similar or dissimilar organisms. One anticipated application would be to create a Stenoprophiluric Agent/Nutrient package which would facilitate and sustain a biofilm/biomass which while being stable in its host habitat for long periods of time would have the ability to “scavenge” or remove specific compounds or pollutants from a host habitat either in a static or flow-by condition. Facilitating the development of certain biomass micro-habitats through management of the settling process or by facilitating the sustained consortium survival thought the availability or lack of availability of specific major or even trace nutrients which control the consortium through their bio-limiting nature is accomplished Stenoprohilurically. Many naturally occurring biochemical bioactive processes in the form of actual chemical change, motive response, trigger of specific activities, or simple communication for attraction or repulsion of agents or organisms which occur at minute concentrations can then be understood and be easily sampled and analyzed for their discreet bio-active roles. Many organisms found in various host habitats, while changing; from planktonic to sessile states of the same organism, exhibit phenotypic expression due to settling, life cycle evolution due to changes in nutrition, reproduction based on availability of shelter or food source. Use of Stenoprophiluric media resulting in larger biomasses and quantities of compounds used in such activities can be studied, collected, and/or manipulated to better understand the causal relationships between specific compounds and/or groups of compounds which alter the biological processes of the organisms involved. Allelopathy, response triggering, communication, attraction, repulsion, alteration of phenotypic growth and disease expression, initiating or ending dormancy, facilitating germination or reproduction, toxification, fertilization, contraception, and/or other metabolic functions of organisms which are influenced amongst consortium participants, create the micro-habitat for study which can provide an understanding of the biochemistry of the interactions. By manipulating differing forms of Stenoprophiluric media or introducing identical media into differing micro-habitats or by varying media composition or architecture and introducing the media to identical habitats, or managing these variable media into manipulated host habitats; study, collection, and bioassay of the mechanisms involved can be accomplished. In providing Stenoprophiluric media changes, specific and targeted biomass or bio-film consortia may be evaluated by modem means of microscopy which permits live imaging. By colonizing usually small micro-habitats into larger colonies, size and numbers are improved which permit study, collection of compounds, and duplication of observed mechanisms to permit verification of discovery at a more focused rate. Microscopic level matrices on small coupons in particular habitats up to and including very large “bio-reactors” where vast surface areas are provided with unique Stenoprophiluric properties which could result in actual “farming” or larger collection of specific compounds and/or materials which could be used as single isolates or in combinations as they generally do in nature are accomplished.

By producing Stenoprophiluric media for specific targeted conditions such as antibiotics from marine sponges or hydroids, platforms of media can be produced which provide the necessary media for the settling or inoculation of a surface which provides selective consortium development of either single or multiple organisms which amongst themselves can trigger measurable responses of biochemical mechanisms or be triggered by host habitat or external stimulation to provide for specific biochemical expression.

Media of similar or dissimilar composition can be placed on any convenient shape including but not limited to; sheets, rods, tubes, cylinders, plates, coupons or discs and introduced singly or in combination to provide for the creation of bio-films or biomasses which may be caused to trigger bioactive responses which may be studied, sampled or otherwise characterized. These shapes may be introduced into host habitats where taxonomic, biochemical, morphological, metabolic, electrochemical, or phenotypic expression may be observed and/or collected and/or measured. Juxtaposition of the media can be accomplished in totally open host environments where sampling and view is accomplished via in-situ methods of direct observation or appropriate sampling methods. Alternatively, semi “open” conditions can be employed where all elements and conditions of the related activities of the Stenoprophiluric biomasses are conducted in a flow through containment system where sampling is facilitated by a semi-enclosure or sampling capture mechanism. Media can be used in partly controlled host habitats where the host habitat is not altered but managed to contain and/or collect metabolic and extra-cellular compounds. Media can be sued in semi controlled environments where all conditions of the host habitat are sustained in a modified host habitat which facilitates the continued or productive formation of a desired expression.

Media can be prepared using any Stenoprophiluric media type; polymer, composite, amalgam, naturally occurring or synthetic materials. The structure of the media can me macroscopically managed or microscopically managed for specific applications where larger organisms such as hydroids, invertebrates, or sessile vertebrates may be used. Or additionally at the microscopic level where physical, chemical, nutritive, or Agent position is used to provide a suitable substrate for settling, formation, and the sustaining of a desired biofilm or micro-colonialization pattern.

Agents may be used singly or in combination where a desired consortium or expression of a desired consortium which includes exo-polymers or extra-cellular materials are used to provide protection or sources of materials and/or compounds which effect bio-activity. This would include cases where the Agent became a component of the exudate or exo-polymer of an organism or consortium of organisms. And/or where specific bio-polymers are produced which have novel bioactive properties. Copper and copper compounds or alloys would provide several of these examples.

Nutrient relationships or combinations of nutrients with the designed purpose of either facilitating a desired settling pattern response, of selected organisms is readily accomplished. Additional trace nutrients and even ultra-trace nutrients can be used to provide a desired consortium or provide for triggers for desired metabolic functions or “raw materials” as “essential nutrients or compounds”

By creating bio-films or micro habitats in situ or semi-controlled habitats, the ability to sample/collect and analyze compounds which are involved with the interaction or production of responses can be recognized, isolated, and measured in isolation or in complexes heretofore not possible in seeing bio active response.

Before discussing specifics of the invention, it is important to point out what the invention is not. The invention is not a classic batch reactor or reaction in which a biomass is used to generate a chemical product in a host habitat fluid, with the reaction proceeding to a point where the biomass and the product are removed and separated from each other in a batch manner. An example of that type of reactor/reaction is the fermentation of sugars in a liquid solution of grain products by yeast to produce beer, wine or other spirits. The biomass is typically not affixed to a substrate there. But even if the bio-mass was affixed, the goal of the fermenter is to take the reaction so far that the alcohol concentration in the host habitat fluid to the level that it becomes toxic to the biomass. The next batch reaction will be carried on by a new biomass. There is no effort to sustain the biomass in an equilibrium condition, which is the touchstone of stenoprophilicity.

With that in mind, attention is directed to FIG. 1, which shows a schematic view of an embodiment of the present invention. The device 10 comprises several elements. First, it has a container 12, to isolate the reaction for control purposes. However, the concept of containment should be broadly understood. A classic container is a solid tube, perhaps made of metal. In fact, it may be made of a metal suitable to serve as a first substrate 14. But in other cases, the container 12 may be an open channel, such as a naturally-occurring stream of water, or any organ of an animal or human that has the appropriate inlet and outlet with fluid flow therethrough. In either case, the container 12 has an inlet 16 and an outlet 18. The container 12 has sufficient structure so that it can contain a host habitat fluid 20, such that the fluid flowably moves from the inlet 16 to the outlet 18. If the container 12 does not have a wall which serves as the first substrate 14, then a separate first substrate is provided, supportably positioned in the host habitat fluid 20. The first substrate 14 is exposed in the container 12 to the host habitat fluid 20, preferably in a manner that a significant stagnant layer is available around the first substrate to keep it from being scoured by the flow. A bio-supportive medium 22, also described elsewhere in this specification as a stenoprophiluric medium, is deposited on the first substrate 14. This bio-supportive medium 22 comprises a degradable material, at least one nutritional source and at least one bio-limiting agent dispersed in the degradable material. The degradable material, the at least one nutritional source, and the at least one bio-limiting agent are provided in appropriate quantities to render the bio-supportive medium capable of supporting formation and development of a biomass of a reactive biological agent therein. The biomass has at least a specific consortium of organisms of the same or different species substantially at equilibrium within its environment or the host habitat fluid 20. The bio-limiting agent (or agents) is selected to control the amount and type of species present in the biomass.

In order to elicit the desired chemical activity of the reactive biological agent in the bio-supportive medium 22, it is necessary to supply the container with at least one stimulative agent in the container. To be efficacious, the stimulative agent needs to be present in the host habitat fluid 20, so that it flows past the bio-supportive medium 22 and is able to diffuse through the interface between the bio-supportive medium 22 and the host habitat fluid 20. The stimulative agent is selected so that proximity of the stimulative agent to the biomass elicits production of the chemical product by the reactive biological agent.

Using the fermentation example cited earlier, grain sugars in liquid solution near yeast would elicit the production of ethanol, so that the grain sugars would be the stimulative agent, the yeast would be the reactive biomass (although not in a bio-supportive medium), and the “mash” would constitute the host habitat fluid, with the ethanol serving as the elicited chemical product.

But not all examples would or should result in a depletion of the stimulative agent. For example, the presence of a protein emitted by a competitor may be a stimulative agent. Likewise, a toxin such as dissolved oxygen could act as a stimulative agent in the case of an anaerobe. Metal ions in solution could also act as stimulative agents.

In many cases, the desired or elicited chemical product will be dispersed from the reactive biological agent into the bio-supportive medium 22, from whence it will diffuse across the interface into the bulk host habitat fluid 20 and be carried out of the container 12 at the outlet 18 by conventional techniques that will be clear once the chemical product and the host habitat fluid 20 are determined.

In other cases, the chemical product generated by the reactive biological agent will collect in the biomass, where it will remain, especially if stored in organelles of the biological agent that actively oppose natural diffusion or osmosis by imposing barriers thereto. In such a case, it will be necessary to, either continuously or on a periodic basis, to cause a portion of the biomass to slough off of the bio-supportive medium 22. This sloughed-off portion will flow through the host habitat fluid 20 and be recovered at the outlet 18. Once recovered, the biomass will have to be processed further to isolate and concentrate the desired chemical product, but, as with the chemical product dispersal situation, this will be clear to one of ordinary skill once the product is determined.

The next issue faced in the invention is placement of the stimulative agent in the host habitat fluid 20 into proximity of the bio-supportive medium 22. There are several options, the optimal selection being mandated by the type of the stimulative agent. In a first case, the stimulative agent may be a component of the host habitat fluid 20 that is introduced directly into the container 12 through the host habitat fluid injected into the inlet 16. This would be appropriate for a stimulative agent that will be depleted in the process, such as the grain sugars in a fermentation. In a second case, the stimulative agent may be better introduced by release from a second substrate 24. One example would be a metal plate in a host habitat fluid appropriate to slowly dissolve the plate, releasing metal ions into the host habitat fluid. Another example of this would be a compound stored in a plurality of micro-capsules on the surface of the second substrate 24, so that degradation of the micro-capsules would release the stimulative agent.

Perhaps most importantly, if the stimulative agent is a biologically-produced compound, such as an identification protein emitted by a microbe, the second substrate 24 will possess a deposited second bio-supportive medium 26, analogous to the first bio-supportive medium 22. In fact, mutual interaction of two competitive bio-supportive media 22, 26 on proximally situated first and second substrates 14, 24 could be an optimal use of the technology of the present invention, as it could result in generation and collection of multiple chemical products.

Once the decision is made as to whether there should be only a first substrate 14 or both a first and a second substrate 14, 24 in the particular system, then the exact selection of placement and shape factors in the substrates 14, 24 can be addressed. Known and desirable shapes include cylinders, plates and the like, in order to maximize surface area per unit volume.

Also very important in the reactor device 10 is the decision as to how to position and maintain the substrates 14, 24 properly to provide the correct proximity necessary to eleicit optimal chemical production. For this reason, each substrate 14, 24, when two substrates are used, should be movable relative to the other.

It should also be clear that the invention is not limited to either a single first substrate 14 or a single second substrate 24.

In one example of the present invention, cylinders are used to support differing stenoprophiluric media. When the media are immersed in host habitat fluid in the container at a desired location, different bio-films are formed. When these bio-films are then brought into close proximity to one another, defense of space and invasion cues are triggered in the bio-films. These cues result in the formation of chemical compounds that are used by the organisms for communication, repellency, and/or other metabolic or extra-cellular functions. To perform these extra-cellular functions, the chemical compounds are not retained in the biomass, but are instead dispersed outwardly, where they diffuse into the host habitat fluid. Since the host habitat fluid flows from the container inlet to the container outlet, the chemical compounds are swept out of the container at the outlet. The compounds can then be collected and evaluated for bio-activity for other purposes.

An alternate method of creating other relationships between biomasses would be to have one consortium on a cylinder and another distinct consortium on another shape like a plate. Light (illumination) dynamics, flow by characteristic of sampling limitations may dictate any shape where the integrity of a biomass is maintained separately and brought into some spatial contact with another community. Space relationships could be very large. Essentially any connection via a host habitat, even at great distances could be used. Whenever a product or by-product of a Stenoprophiluric community influences another either natrual or Stenoprophiluric community the benefit of the at least one Stenoprophiluric community is realized.

Similarly, panels which are 12″ by 12″ squares can be arranged to support variations in Stenoprophiluric media. In a similar fashion, once an initial, or established biofilm or biomass community is formed, placing the panels in proximity triggers biochemical responses.

In a like fashion, tubes can be placed in a system where Stenoprophiluric media are affixed to the interior surface. In this example the interior surface is used to provide the location for the biofilm or biomass development. After dissimilar biofilms or masses are initiated or established, they then can share a common stream or flow through creating another form of triggering, “downstream” from one another. This method is akin to stream ecology and dynamics which can be utilized in flowing systems.

Any system which provides for dissimilar biofilm or biomass creation which are then brought into juxtaposition to effectively mimic invasion, settlement, or intrusion of “space” can be employed. The objective is to cause a relationship between two different biological communities whereby metabolic and allelochemical reactions will occur which may be studied or sampled including the generated chemical compounds, proteins, and/or communication means for initializing biological and chemical responses.

Another method involves rotating drums in differing host habitats either natural or synthetic. Drums of any nominal size, depending on the availability of space, such as 12 inch diameter cylinders that are 24 inches long are each coated independently with a different Stenoprophiluric media. One cylinder could be coated with a copper/epoxy system while the second cylinder is coated with an iron/epoxy system or a zinc/epoxy system. The cylinders are placed in the host habitat, such that they may be rotated in a horizontal plane so that the biofilm or biomass that forms on the cylinder over time comes in proximal or adjacent contact sufficient that biochemical communication and or allelopathic responses to the other biomass results in biochemical change. This method provides the opportunity for the continuous sampling of either the released chemical compounds, the released biomass components, or products from the biomass of any form to be collected via suitable sampling devices. This sampling may include periodic sampling of the biomass down to the substrate of the cylinders where biofilm or biomass communities form inter-related morphological communities with biochemical activity at the various levels of the relationships of the components in metabolic, spatial settling, nutrition, or other biological activity which results in chemical, physical, or biological changes or reactions to the Stenoprophiluric system which can provide for the identification of novel compounds for any purpose including pharmacological and fine chemicals.

Any situation where potentially opposing or competing biomasses can be brought in to proximity once they are established as independent consortiums where the new relationship for competition for food, space or light or other need is affected, change may be triggered, this change may be for the purpose of communication, repellency, or allelopathic in nature. Having sufficient quantity of these unique compounds produced under this directed circumstance provides for the novelty of this application.

Additional attributes of Stenoprophiluric media in the study of in-situ bio-chemical conditions in micro-habitats include settling management, sustained micro-habitats, is the ability to stage availably as a function of the Stenoprophiluric media management over time. From the previously described macro-level deployment of Stenoprophiluric media additional examples of micro-level control of the media and resultant consortiums can be accomplished.

In developing micro-habitats for evaluation of bio-chemical behavior and resultant compounds, settling can be managed by the spatial deployment of nutrients and Stenoprophiluric agents. Adjusting particle size or molecule size of the media can facilitate the selection of specific organisms by providing a spatial electrical “grid” pattern or a nutritional “grid” pattern which can be attractive to one set of organism(s) and provide improved conditions for attachment and deployment to a second and third stage of community development. In copper epoxy systems changing the size of a copper particulate in a Stenoprophiluric coating from 50 microns to 15 microns will modify the consortium dramatically. At 50 microns the consortium predominates in Algal species, where at 15 microns bacterial species initialize as the predominant species. This has been attributed to at least three relationships singly or in combination. A spatial electrically charged variant surface between the base media and particulate which is occluded or integral to the polymeric matrix. The nutritional availability of the spatially deployed copper in specific species and concentration as afforded by the matrix, and the nutrition package which is afforded by the matrix of the Stenoprophiluric material which contains nutritional and interactive Agents for the management of the consortium.

Similarly a polymer can be altered to provide differing structure by adjusting the molecular weights of molecules and monomers in assembly of a polymer. The resultant polymer creates a differing spatial periodicity which at certain ends or nodes profvides a prescribed electrical, chemical, or nutrient pattern which facilitates settling or selection or predominance of a certain biomass or consortium over time. This effect may be changed through the depth of the Stenoprophiluric media in order to cause a timed change.

In sustaining micro habitats beyond a few weeks and even into years as much as 20 years, along with basic nutrients as supplied by the media and the host habitat, another manner in which a Stenoprophiluric media may provide differentiation of consortiums or the ability to sustain such a consortium is related to trace or even ultra-trace concentrations of nutrients and/or Agents. By dispersing such elements as selenium or zinc or other bio-limiting agents either positively or negatively can influence both primary selling, affect exponential growth capability, and/or long term sustained micro-habitats based on the availability of such controlling chemicals, elements, or compounds. It must be noted that this effect can be also affected by universal distribution of the Agent or nutrient in the matrix or by subsequent layering which would provide the species and availability of the agent or compound in a time sequence dependent on the matrix location. In one demonstration a Stenoprophiluric matrix was formed by the sequential layering of differing matrices, one on top of the other. A base Stenoprophiluric matrix of epoxy and copper was applied to a substrate where the layers of base coating were applied in increments of 1 to 2 mils. (thousandths of an inch) where a “flash” coat of the same matrix with the addition of Selenium at a concentration of 10 to 50 part per million was applied in a layer which did not exceed 0.5 mils. Was applied between successive layers of the base coating. This provided the availability of the Selenium at differing locations in the final layered matrix. Consequential management of the surface over time by altering the chemical, electrical, and nutritional composition of the matrix provides for a management tool related to time for controlling the resultant biomass and subsequent changes due to the interaction with the matrix. This method is usable with any Agent or Nutrient when building a Stenoprophiluric media. The only limitation is that the Agent or Nutrient must be in a form (species) and concentration that is mediated by the desired biomass or bio-film micro-habitat consortium desired interaction.

In another application, panels of Stenoprophiluric coatings can be assembled which are 4′ by 4′ square whereby the panels are exposed in a host habitat and either the sampled or collected on some regular basis, in essence farming the resultant biomass. This collection method would be completed in such a manner that components or combinations of components as chemicals, compounds or materials are used to provide models for development of compounds or chemical compositions or themselves be the source of such compounds for study or commercial use.

1. A device for isolating a chemical product generated by proximity of a reactive biological agent to a stimulative agent, said device comprising:
a container with an inlet and an outlet;
a host habitat fluid, flowably moving through the container from the inlet to the outlet;
at least one first substrate, supportably positioned in the container between the inlet and the outlet thereof;
a bio-supportive medium, with at least one layer thereof deposited on each said at least one first substrate, the bio-supportive medium comprising a degradable material, at least one nutritional source and at least one bio-limiting agent dispersed in the degradable material, wherein the degradable material, the at least one nutritional source, and the at least one bio-limiting agent are provided in quantities, such that the bio-supportive medium is capable of supporting formation and development of a biomass of the reactive biological agent therein, the biomass having at least a specific consortium of organisms of the same or different species substantially at equilibrium within its environment or host habitat fluid, wherein the at least one bio-limiting agent is selected to control the amount and type of species present in the biomass; and
at least one stimulative agent in the container, the stimulative agent selected such that proximity of the stimulative agent to the biomass elicits production of the chemical product by the reactive biological agent.

2. The device of claim 1, wherein the chemical product is dispersed by the reactive biological agent into the host habitat fluid and a means for collecting and isolating the chemical product from the host habitat is provided at the outlet.

3. The device of claim 2, wherein the stimulative agent is present in the host habitat fluid.

4. The device of claim 2, further comprising:
at least one second substrate, supportably positioned in the container between the inlet and the outlet thereof and movable therein relative to the at least one first substrate;
wherein the stimulative agent is affixed to the at least one second substrate and is released therefrom in a time-controlled manner.

5. The device of claim 2, further comprising:
at least one second substrate, supportably positioned in the container between the inlet and the outlet thereof and movable therein relative to the at least one first substrate; and
a second bio-supportive medium, with at least one layer thereof deposited on each said at least one second substrate, the second bio-supportive medium comprising a degradable material, at least one nutritional source and at least one bio-limiting agent dispersed in the degradable material, wherein the degradable material, the at least one nutritional source, and the at least one bio-limiting agent are provided in quantities, such that the second bio-supportive medium is capable of supporting formation and development therein of a second biomass that generates and disperses the stimulative agent, the second biomass having at least a specific consortium of organisms of the same or different species substantially at equilibrium within its environment or host habitat fluid, wherein the at least one bio-limiting agent is selected to control the amount and type of species present in the biomass.

6. The device of one of claims 3 through 5, wherein the at least one first substrate is a plate.

7. The device of one of claims 3 through 5, wherein the at least one first substrate is a cylinder.

8. The device of one of claims 3 through 5, wherein the at least one first substrate is a wall of the container.

9. The device of claim 1, wherein the chemical product collects in the biomass and a means is provided at the outlet for collecting and isolating a portion of the biomass that sloughs off of the bio-supportive medium.

10. A process for isolating a chemical product generated by proximity of a reactive biological agent to a stimulative agent, the process comprising the steps of:
providing a host habitat fluid in a container having an inlet and outlet, such that the host habitat flows from the inlet to the outlet;
positioning at least one first substrate in the container between the inlet and the outlet, the at least one first substrate having a bio-supportive medium, with at least one layer thereof deposited on each said at least one first substrate, the bio-supportive medium comprising a degradable material, at least one nutritional source and at least one bio-limiting agent dispersed in the degradable material, wherein the degradable material, the at least one nutritional source, and the at least one bio-limiting agent are provided in quantities, such that the bio-supportive medium is capable of supporting formation and development of a biomass of the reactive biological agent therein, the biomass having at least a specific consortium of organisms of the same or different species substantially at equilibrium within its environment or host habitat fluid, wherein the at least one bio-limiting agent is selected to control the amount and type of species present in the biomass;
providing further amounts of the at least one nutritional source to the biomass through the host habitat fluid;
introducing at least one stimulative agent in the container, the stimulative agent selected such that proximity of the stimulative agent to the biomass elicits production of the chemical product by the reactive biological agent; and
collecting and isolating the chemical product at the outlet of the container.

11. The process of claim 10, wherein the chemical product is dispersed by the reactive biological agent into the host habitat fluid and the collecting and isolating step removes the chemical product directly from the host habitat fluid.

12. The process of claim 10, wherein the chemical product collects in the biomass and the collecting and isolating step removes from the host habitat fluid a portion of the biomass that sloughs off of the bio-supportive medium.
